(19) United States
(12) Patent Application Publication
(10) Pub. No.: US 2026/0224269 A1
(43) Pub. Date: Aug. 6, 2026

(54) HIGH FREQUENCY OUTPUT DEVICE, HANDPIECE THEREFOR, AND TIP COUPLABLE TO HANDPIECE

(71) Applicant: WONTECH CO., LTD., Daejeon (KR)

(72) Inventors: Jong Won KIM, Seongnam (KR); Jung Hyun KIM, Seongnam (KR); YoungSeok SEO, Seiong (KR); Jung Il PARK, Seongnam (KR); Ye Chan NAM, viwang (KR)

(21) Appl. No.: 19/204,682

(22) Filed: May 12, 2025

(30) Foreign Application Priority Data
Feb. 6, 2025 (KR) 10-2025-0015002

(51) Int. Cl. A61B 18/08; A61B 18/00

(52) U.S. Cl. CPC A61B 18/082; A61B 2018/00023; A61B 2018/00178; A61B 2018/00452

(57) ABSTRACT

Proposed is a tip couplable to a handpiece. A tip according to an embodiment includes a housing, a frame coupled to the housing, an electrode module coupled to the frame and outputting electrical energy, and a cooling module spraying a refrigerant into a first space at least partially partitioned by the frame, wherein the electrode module includes a substrate, and an electrode formed on a region of the substrate and outputting the electrical energy, and the refrigerant is introduced from the handpiece into the cooling module and then sprayed toward the region of the substrate. According to the embodiment, it is possible to provide an effective cooling structure capable of cooling a tip or a treatment site during treatment with a high frequency output device.

### CROSS REFERENCE TO RELATED APPLICATION

The present application claims priority to Korean Patent Application No. 10-2025-0015002, filed February 6, 2025, the entire contents of which is incorporated herein for all purposes by this reference.

### BACKGROUND OF THE INVENTION

### Field of theInvention

The present disclosure relates generally to a high frequency output device, a handpiece therefor, and a tip couplable to the handpiece. More particularly, the present disclosure relates to a high frequency output device that outputs electrical energy to human body tissue for skin care or medical purposes, a handpiece therefor, and a tip used by coupling to the handpiece.

### Description of the Related Art

With the advancement of medicine, devices that improve human skin tissue using various energy sources are being developed.

Among them, a high frequency output device is a device that uses electrical energy as an energy source. The high frequency output device emits electrical energy of a predetermined frequency (e.g., radio frequency) toward the tissue under the human skin to increase the temperature inside the skin, thereby reorganizing the collagen layer within the tissue, increasing skin elasticity, and reducing wrinkles.

During treatment using the high frequency output device, a patient may feel pain and may experience skin burns due to extreme heat generated from a tip. To solve the above problems, the skin of the patient can be cooled with ice before or during the treatment, thereby reducing the risk of pain and burns during the treatment. Alternatively, the problems can be alleviated by cooling the temperature of the tip that outputs electrical energy.

However, the related art does not propose an effective cooling technique that can be utilized during the treatment. For example, a method of cooling a treatment site using ice is problematic in that the treatment needs to be temporarily stopped to cool the treatment site with an ice bag. Another cooling method involves using a cooling device that sprays a refrigerant through a cooling nozzle. In this case, an assistant next to an operator holds the cooling nozzle and sprays the refrigerant toward the treatment site. Despite the advantage of enabling cooling during the treatment, this method has the disadvantage of requiring a separate cooling device and requiring a separate assistant to spray the refrigerant.

Meanwhile, in terms of cooling, it is important to directly cool the treatment site to prevent burns and pain, but it is also necessary to cool the tip itself, whose temperature has risen during the treatment. This is because lowering the temperature of the tip through cooling can reduce the heat transferred to the skin surface, reduce the risk of pain and burns, and prevent tip malfunction caused by overheating.

The foregoing is intended merely to aid in the understanding of the background of the present disclosure, and is not intended to mean that the present disclosure falls within the purview of the related art that is already known to those skilled in the art.

### SUMMARY OF THE INVENTION

Accordingly, the present disclosure has been made keeping in mind the above problems occurring in the related art, and one objective of the present disclosure is to provide a high frequency output device capable of cooling a treatment site and/or a tip during treatment with the high frequency output device, a handpiece therefor, and a tip couplable to the handpiece.

Another objective of the present disclosure is to provide a high frequency output device capable of increasing a cooling effect and uniformly lowering the temperature of a tip by spraying a refrigerant in a distributed manner to a high-temperature area of the tip, a handpiece therefor, and a tip connectable to the handpiece.

Still another objective of the present disclosure is to provide a high frequency output device capable of preventing a phenomenon in which a residual refrigerant liquefies and accumulates inside a tip by rapidly evaporating or discharging the residual refrigerant inside the tip, a handpiece therefor, and a tip connectable to the handpiece.

The technical problems of the present disclosure are not limited to those mentioned above, and other technical problems not mentioned will be clearly understood by those skilled in the art from the description below.

In order to achieve the above objectives, according to one aspect of the present disclosure, there is provided a tip couplable to a handpiece, the tip including: a housing: a frame coupled to the housing; an electrode module coupled to the frame and outputting electrical energy of a predetermined frequency; and a cooling module spraying a refrigerant into a first space at least partially partitioned by the frame. The electrode module may include: a substrate; and an electrode formed on a region of the substrate and outputting the electrical energy toward an outside of the housing. The refrigerant may be introduced from the handpiece into the cooling module and then sprayed toward the region of the substrate.

According to another aspect of the present disclosure, there is provided a high frequency output device, including: a main body; a handpiece connected to the main body; and a tip coupled to the handpiece and receiving electrical energy from the handpiece and outputting the electrical energy toward human body tissue while being coupled to the handpiece. The tip may include: a housing: a frame coupled to the housing; an electrode module coupled to the frame and outputting electrical energy of a predetermined frequency; and a cooling module spraying a refrigerant into a first space at least partially partitioned by the frame. The electrode module may include: a substrate; and an electrode formed on a region of the substrate and outputting the electrical energy toward the human body tissue. The refrigerant may be introduced from the handpiece into the cooling module and then sprayed toward the region of the substrate.

According to still another aspect of the present disclosure, there is provided a handpiece for a high frequency output device, the handpiece including: a tip; an electrical circuit providing electrical energy of a predetermined frequency to the tip; and a controller controlling an operation of the electric circuit. The tip may include: a housing: a frame coupled to the housing; an electrode module coupled to the frame and outputting electrical energy of a predetermined frequency; and a cooling module spraying a refrigerant into a first space at least partially partitioned by the frame. The electrode module may include: a substrate; and an electrode formed on a region of the substrate and outputting the electrical energy toward an outside of the housing. The refrigerant may be introduced from the handpiece into the cooling module and then sprayed toward the region of the substrate.

According to the present disclosure, it is possible to provide an effective cooling structure capable of cooling a tip or a treatment site during treatment with a high frequency output device.

Additionally, by spraying a refrigerant in a distributed manner to a high-temperature area of the tip, it is possible to increase a cooling effect and uniformly lower the temperature of the tip.

Additionally, by providing one or more channels that communicate spaces inside the tip or communicate the inside of the tip with the outside of the tip, it is possible to quickly evaporate or discharge residual refrigerant inside the tip, thereby effectively preventing a phenomenon in which the residual refrigerant liquefies and accumulates inside the tip.

The effects of the present disclosure are not limited to those mentioned above, and other effects not mentioned will be clearly understood by those skilled in the art from the description below.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a perspective view illustrating the configuration of a high frequency output device according to an embodiment of the present disclosure;

FIG. 2A and FIG. 2B are a perspective projection view exemplarily illustrating a tip according to an embodiment of the present disclosure;

FIG. 3 is a view exemplarily illustrating the main configuration of the tip according to an embodiment of the present disclosure;

FIG. 4A and FIG. 4B are an exemplary view illustrating in detail the configuration of a housing illustrated in FIG. 3;

FIG. 5A and FIG. 5B are an exemplary view illustrating in detail the configuration of a base member illustrated in FIG. 3;

FIGS. 6A, 6B and 7 are exemplary views illustrating in detail the configuration of a cooling module illustrated in FIG. 3;

FIG. 8A and FIG. 8B are an exemplary view illustrating in detail the configuration of an electrode module illustrated in FIG. 3;

FIG. 9A and FIG. 9B are an exemplary view illustrating in detail the configuration of a cover member illustrated in FIG. 3;

FIG. 10 is a view illustrating an exemplary assembly method of the tip according to an embodiment of the present disclosure;

FIG. 11 is a view illustrating an internal space of the tip, in which the internal space is partitioned by the main components of the tip;

FIGS. 12A, 12B, 12C and 12D are a view illustrating various embodiments in which a fluid channel is formed inside the tip to more effectively remove a residual refrigerant inside the tip; and

FIG. 13 is a graph illustrating the results of an experiment comparing the difference between the case where a packing member is applied and the case where no packing member is applied.

### DETAILED DESCRIPTION OF THE INVENTION

Reference will now be made in greater detail to exemplary embodiments of the present disclosure with reference to the accompanying drawings. The advantages and features of the present disclosure, and objectives achieved by the present disclosure will become apparent from the detailed description of the following embodiments in conjunction with the accompanying drawings. It should be understood that the present disclosure is not limited to the following embodiments and may be embodied in different ways, and that the embodiments are given to provide complete disclosure of the present disclosure and to provide a thorough understanding of the present disclosure to those skilled in the art. The scope of the present disclosure is defined only by the claims.

As for reference numerals associated with elements in the drawings, it should be noted that the same elements in different drawings are denoted by the same reference numerals. Further, in the following description of the present disclosure, a detailed description of known functions and configurations incorporated herein will be omitted when it is determined that the description may make the subject matter of the present disclosure rather unclear.

Unless otherwise defined, all terms including technical and scientific terms used herein have the same meaning as commonly understood by one of ordinary skill in the art to which the present disclosure belongs. It will be further understood that terms, such as those defined in commonly used dictionaries, will not be interpreted in an idealized or overly formal sense unless expressly so defined herein. The terminology used herein is for the purpose of describing particular embodiments only and is not intended to be limiting. As used herein, the singular forms are intended to include the plural forms as well, unless the context clearly indicates otherwise.

Further, terms, such as first, second, A, B, (a), or (b) may be used herein to describe elements of the disclosure. Each of these terms is not used to define essence, order, sequence, or number of an element, but is used merely to distinguish the corresponding element from another element. When it is mentioned that an element is “connected”, “coupled”, or “linked” to another element, it should be interpreted that another element may be “interposed” between the elements or the elements may be “connected”, “coupled”, or “linked” to each other via another element as well as that one element is directly connected or coupled to another element.

Hereinbelow, some embodiments of the present disclosure will be described in detail with reference to the accompanying drawings.

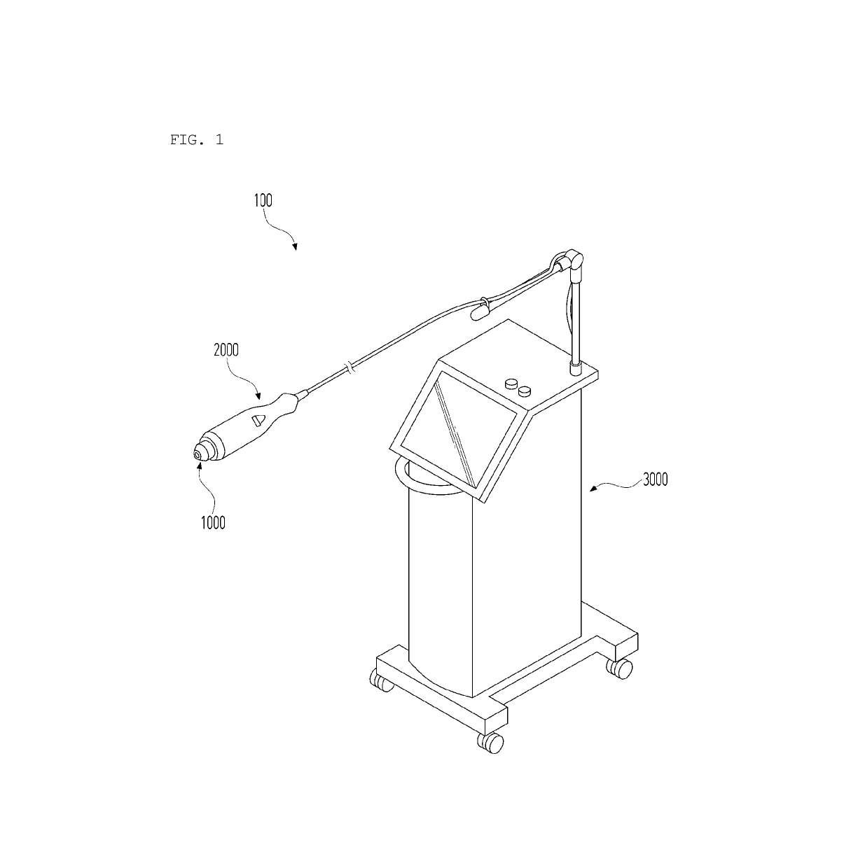

FIG. 1 is a perspective view illustrating the configuration of a high frequency output device 100 according to an embodiment of the present disclosure.

Referring to FIG. 1, the high frequency output device 100 may include a tip 1000, a handpiece 2000, and a main body 3000.

The main body 3000 generates electrical energy of a predetermined frequency (e.g., radio frequency) required for skin treatment. At this time, the generated electrical energy may be output in the form of electromagnetic waves or electric current and emitted onto the human skin.

In this case, the frequency of the electrical energy may be determined differently depending on patient’s treatment purpose or treatment site. For example, for skin treatment purposes, the electrical energy may have a frequency of 0.1 MHz to 0.8 MHz.

In an embodiment, the main body 3000 may include a power supply unit (not illustrated) that generates electrical energy, a regulator unit (not illustrated) that adjusts the output of the electrical energy, a modulation unit (not illustrated) that adjusts the frequency of the electrical energy, a display unit (not illustrated) that displays the overall status of the high frequency output device 100 or patient information and provides a user interface for operating the high frequency output device 100, and/or a control unit (not illustrated) that controls the overall operation of each of the above units.

In an embodiment, the main body 3000 may include a refrigerant supply portion (not illustrated) that provides a refrigerant for cooling the tip 1000. The refrigerant supply portion may accommodate a refrigerant can, and may withdraw the refrigerant contained in the refrigerant can at an appropriate timing and provide it to the handpiece 2000 and/or the tip 1000. At this time, the refrigerant may be, but is not limited to, a cryogen in liquid or gaseous form.

The handpiece 2000 may have the tip 1000 detachably attached to an end thereof, and may transmit electrical energy provided from the main body 3000 to the tip 1000, thereby allowing the electrical energy to be output to the skin through the tip 1000.

The handpiece 2000 may include an electric circuit (not illustrated) that provides electric energy of a predetermined frequency to the tip 1000 and a controller (not illustrated) that controls the operation of the electric circuit, and may control the overall operation of the tip 1000.

For example, an outer surface of the handpiece 2000 may be provided with one or more operating buttons, and the controller may control the tip 1000 to output or not output the electrical energy depending on on/off of the operating buttons. Alternatively, depending on the operation of the operating buttons, the controller may adjust the intensity or frequency of the electrical energy output by the tip 1000, or adjust the amount or injection timing of the refrigerant injected into the tip 1000.

In an embodiment, the handpiece 2000 may include a refrigerant injection port (not illustrated). The refrigerant injection port is a configuration for allowing the refrigerant for cooling to be injected into the tip 1000. For example, the handpiece 2000 may receive the refrigerant from the refrigerant supply portion of the main body 3000 and inject the received refrigerant into the tip 1000 using a pump or valve, etc.

The tip 1000 may receive the electrical energy from the handpiece 2000 and output it to the human skin through an electrode module (not illustrated). At this time, the outputting of the electrical energy may achieved by emitting the electrical energy in the form of electromagnetic waves toward the human skin or by applying the electrical energy in the form of electric current. The tip 1000 may be a non-invasive type tip that outputs the electrical energy in a non-invasive manner while the electrode module is in close contact with the surface of the human skin, but the scope of the present disclosure is not limited thereto. For example, the tip 1000 may be an invasive type tip that is provided with a micro needle array connected to the electrode module and outputs the electrical energy through a distal end of the needle while the micro needle array penetrates the human skin.

The tip 1000 may include a cooling module (not illustrated) that sprays the refrigerant injected from the handpiece 2000 to the inside or outside of the tip 1000. The cooling module may spray the refrigerant injected from the handpiece 2000 toward the electrode module, thereby effectively cooling the electrode module, which gradually becomes heated as the electrical energy is output. Meanwhile, although this specification describes an embodiment in which the cooling module sprays the refrigerant toward the inside of the tip 1000, the scope of the present disclosure is not limited thereto. For example, the cooling module may further include a nozzle extending therefrom and oriented toward the treatment site, and may perform direct cooling of the treatment site by spraying the refrigerant through the nozzle toward the treatment site.

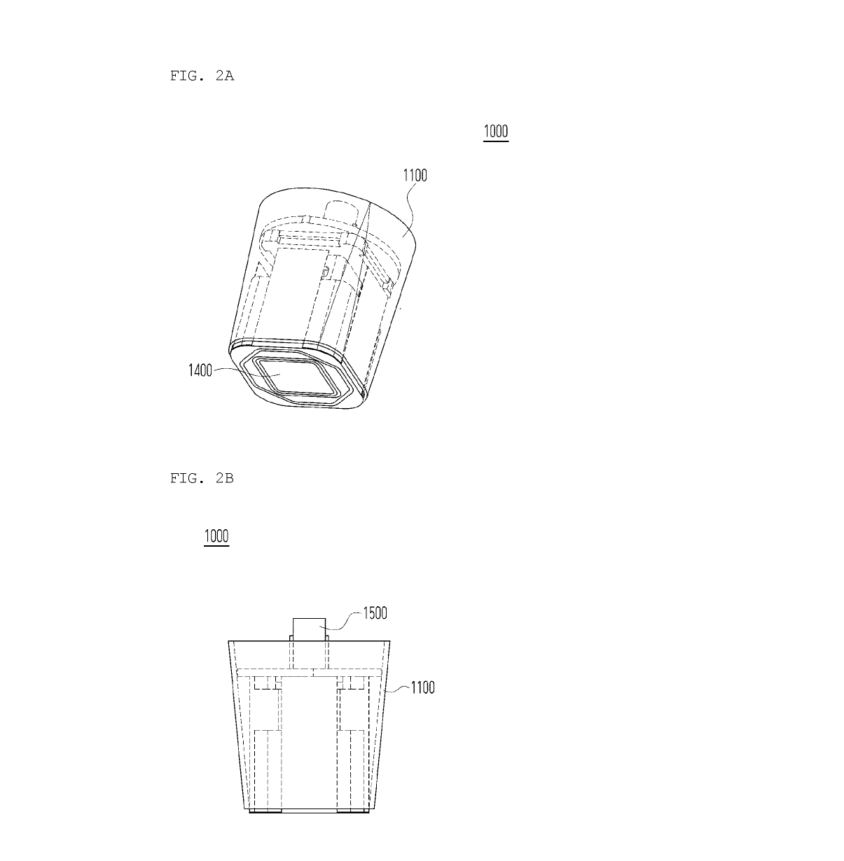

Hereinbelow, the detailed configuration and operation of the tip 1000 will be described in more detail with reference to FIG. 2A and FIG. 2B and thereafter.

FIG. 2A and FIG. 2B are a perspective projection view exemplarily illustrating a tip 1000 according to an embodiment of the present disclosure. Referring to FIG. 2A and FIG. 2B, the tip 1000 may externally include a housing 1100 and an electrode module 1400.

The housing 1100 is a configuration that provides a mechanical casing for the tip 1000 and functions to protect components of the tip 1000 from various external forces or impacts. The housing 1100 may have, but is not limited to, a cylindrical shape with a first end and/or a second end open.

The electrode module 1400 may output electrical energy transmitted from the handpiece 2000 in the form of electromagnetic waves or electric current. At least a portion (e.g., an electrode portion) of the electrode module 1400 may be located at the first end of the housing 1100 and exposed to the outside. During treatment, the exposed portion of the electrode module 1400 may be brought into close contact with the human skin, and in that state, the electrical energy may be output through the exposed portion.

In an embodiment, a cover member 1500 may be provided at the second end of the housing 1100. The cover member 1500 may fix the position of each of the components of the tip 1000 so that the components are stably stored inside the housing 1100. Additionally, the cover member 1500 may also function to guide electrical connection and/or refrigerant delivery between the handpiece 2000 and the tip 1000. Since the cover member 1500 is located at a joint between the tip 1000 and the handpiece 2000, the cover member 1500 may not be exposed to the outside while the tip 1000 is coupled to the handpiece 2000.

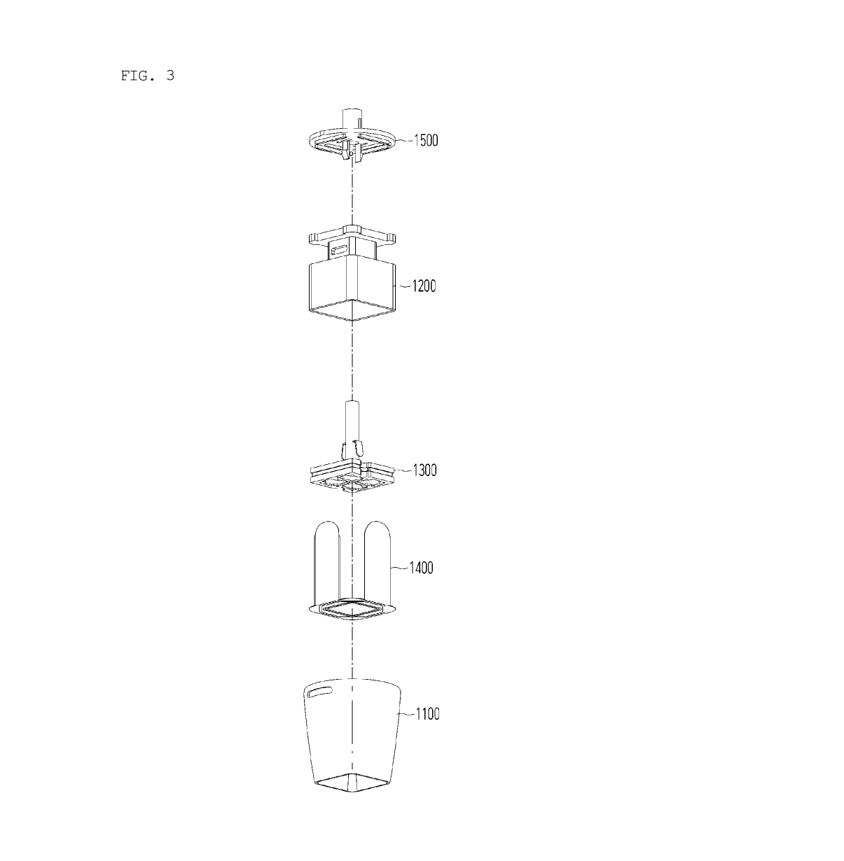

FIG. 3 is a view exemplarily illustrating the main configuration of the tip 1000 according to an embodiment of the present disclosure. Referring to FIG. 3, the tip 1000 may include a housing 1100, a base member 1200, a cooling module 1300, an electrode module 1400, and a cover member 1500.

The base member 1200 and the cover member 1500 may be components constituting a frame 1200, 1500. That is, the base member 1200 and the cover member 1500 may be coupled to each other to form the frame 1200, 1500. In this case, the frame 1200, 1500 may refer to one module including the base member 1200 and/or the cover member 1500.

Meanwhile, in the present embodiment, the base member 1200 and the cover member 1500 are exemplified as being individual elements that are distinct from each other, but the scope of the present disclosure is not limited thereto. For example, a single frame manufactured as an integrated body may replace the base member 1200 and the cover member 1500.

Referring to FIG. 3, the housing 1100 provides a casing for the other components 1200, 1300, 1400, and 1500.

The base member 1200 and the cover member 1500 may be coupled to each other to form the frame 1200, 1500, and the frame 1200, 1500 may be coupled to the housing 1100.

The electrode module 1400 may include a substrate and an electrode. The substrate may include an electrode region located centrally and one or more legs extending from the electrode region. The electrode may be disposed on the electrode region to output electrical energy of a predetermined frequency through an open end of the housing 1100. The one or more legs may be made of a flexible material, and may be fitted to the frame 1200, 1500 to secure the electrode module 1400 to the frame 1200, 1500.

The cooling module 1300 may spray a refrigerant into a first space at least partially partitioned by the frame 1200, 1500, for example, a space surrounded by the base member 1200 and the electrode module 1400. At this time, the refrigerant may be sprayed toward the electrode region of the electrode module 1400, and after being sprayed, the refrigerant may temporarily stay in the first space and cool the electrode region and/or its surrounding region.

As illustrated in FIG. 3, the cooling module 1300 may preferably be accommodated in the base member 1200 while being aligned with the cover member 1500. This simplifies a refrigerant delivery path and an associated mechanical design. However, the scope of the present disclosure is not limited thereto. For example, it is also possible to design the cooling module 1300 to be partially or completely exposed to the outside of the base member 1200.

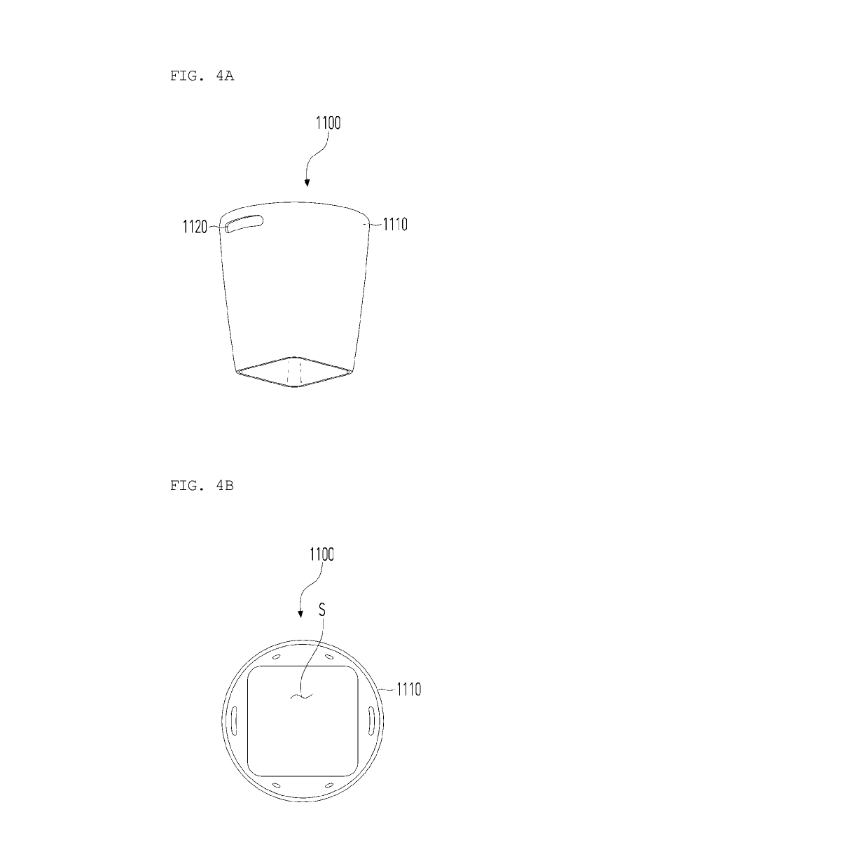

FIG. 4A and FIG. 4B are an exemplary view illustrating in detail the configuration of the housing 1100 illustrated in FIG. 3.

Referring to FIG. 4A and FIG. 4B, the housing 1100 may have a cylindrical shape with a space S formed therein and a first end and a second end open.

The housing 1100 may include a body portion 1110 and one or more tip fastening portions 1120 formed on a side surface of the body portion 1110.

The body portion 1110 forms a side wall of the housing 1100. The body portion 1110 may be made of metal, plastic, or a combination thereof.

The tip fastening portions 1120 are a configuration for fastening the tip 1000 to the handpiece 2000, and may be in the form of perforated holes, but the scope of the present disclosure is not limited thereto. For example, the tip fastening portions 1120 may be in the form of non-perforated dents that are formed by partially cutting an inner wall of the body portion 1110.

Under this configuration, the handpiece 2000 may be provided with one or more hooks or protrusions, and the tip 1000 may be fastened to the handpiece 2000 in such a manner that the hooks or protrusions are engaged with the tip fastening portions 1120 of the tip 1000.

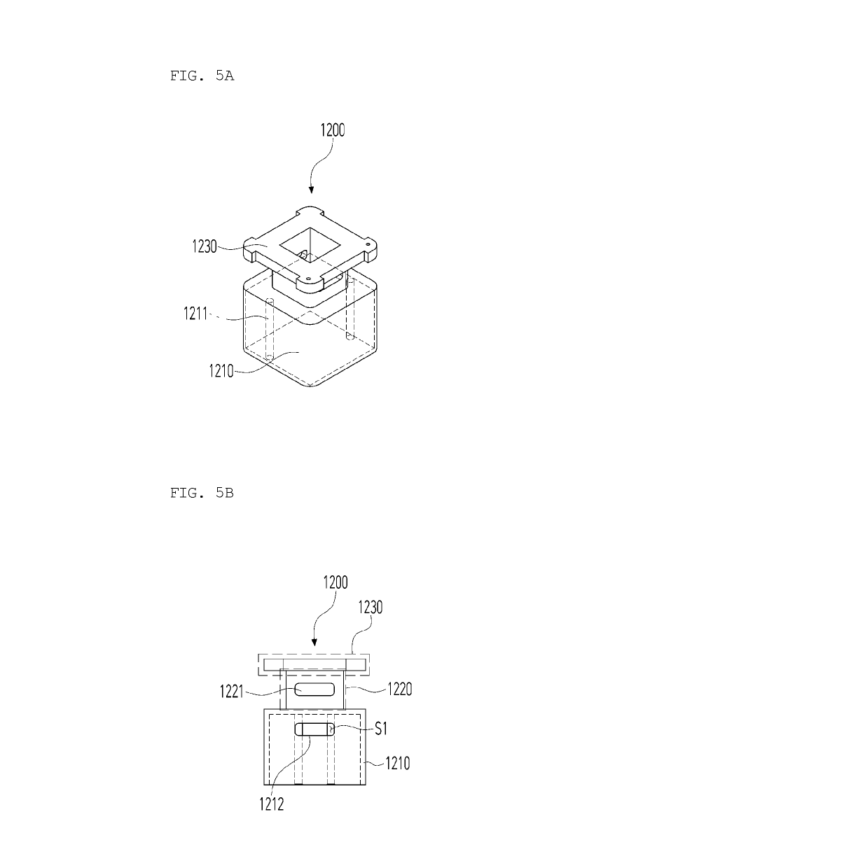

FIG. 5A and FIG. 5B are an exemplary view illustrating in detail the configuration of the base member 1200 illustrated in FIG. 3.

The base member 1200 may be coupled to the cover member 1500 to form the frame 1200, 1500, and the frame 1200, 1500 may be fixedly coupled to the housing 1100.

Referring to FIG. 5A and FIG. 5B, the base member 1200 may include a receptacle 1210, a connecting portion 1220, and/or a cover support portion 1230.

The receptacle 1210 may have a cavity formed therein, and the cooling module 1300 may be accommodated in the cavity. At this time, one or more guide bars 1211 may be formed on an inner wall of the receptacle 1210 to guide the position of the cooling module 1300.

The receptacle 1210 may at least partially define a first space S1 in which the refrigerant sprayed by the cooling module 1300 stays. For example, a space surrounded by the receptacle 1210 and the electrode module 1400 may be defined as the first space S1, and the cooling module 1300 may spray the refrigerant toward the first space S1. After being sprayed, the refrigerant may temporarily stay in the first space S1 and cool the electrode module 1400 and/or its surrounding components.

At least one first channel 1212 may be formed on a surface of the receptacle 1210. The first channel 1212 is a configuration for communicating fluid to or from the receptacle 1210 and may be, for example, a perforated hole. As illustrated in FIG. 5A and FIG. 5B, the first channel 1212 may be formed on a side surface of the receptacle 1210, but the scope of the present disclosure is not limited thereto. For example, the first channel 1212 may be formed on an upper surface of the receptacle 1210.

The connecting portion 1220 may extend from the receptacle 1210 toward the cover support portion 1230, and may have a cylindrical shape with a narrower cross-sectional area than the receptacle 1210. A frame fastening portion 1221 may be formed on a side surface of the connecting portion 1220 to allow the cover member 1500 and/or the cooling module 1300 to be fastened to the base member 1200.

In an embodiment, the frame fastening portion 1221 may be a perforated hole or a non-perforated dent. In this case, a hook or protrusion may be formed on the cover member 1500 and/or the cooling module 1300, and the cover member 1500 and/or the cooling module 1300 may be fastened to the base member 1200 in such a manner that the hook or protrusion is engaged with the frame fastening portion 1221.

In an embodiment, the first channel 1212 may be formed in the connecting portion 1220. In this case, a perforated hole may be formed at an arbitrary position in the connecting portion 1220 to serve as the first channel 1212. Alternatively, when the frame fastening portion 1221 is in the form of a perforated hole, the frame fastening portion 1221 itself may serve as the first channel 1212 without forming a separate hole.

The cover support portion 1230 is a configuration for supporting the cover member 1500, may face the cover member 1500, and may have a shape conforming to the shape of the cover member 1500. For example, when the cover member 1500 has a planar plate shape, the cover support portion 1230 may also have a planar plate shape conforming thereto. Alternatively, when the cover member 1500 has a ball shape, the cover support portion 1230 may have a concave plate shape for accommodating the ball.

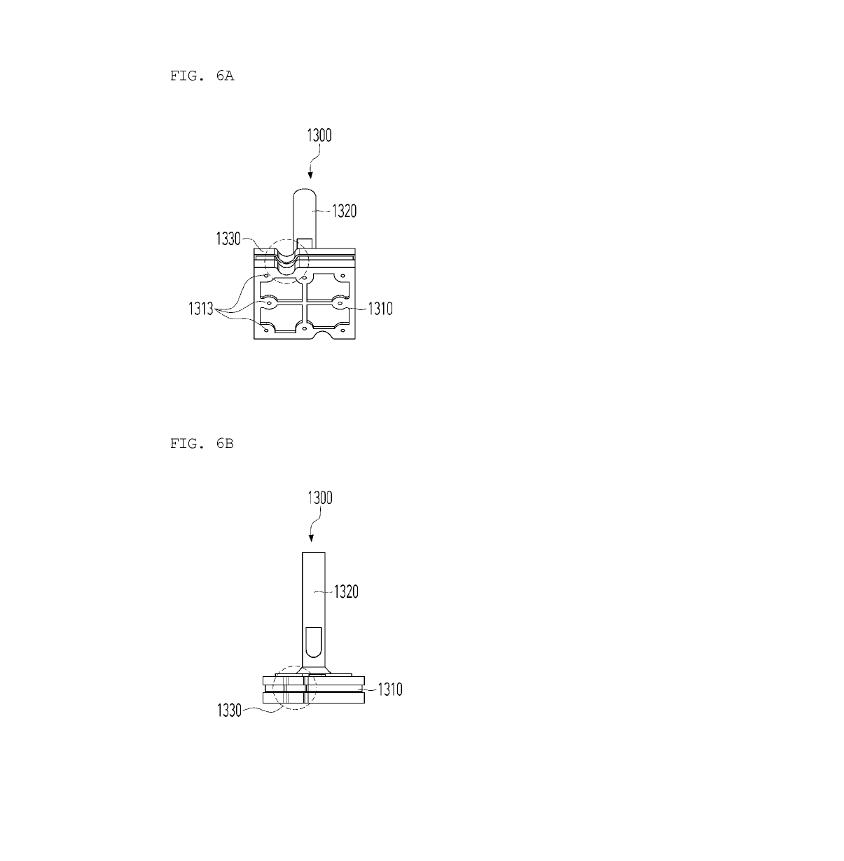

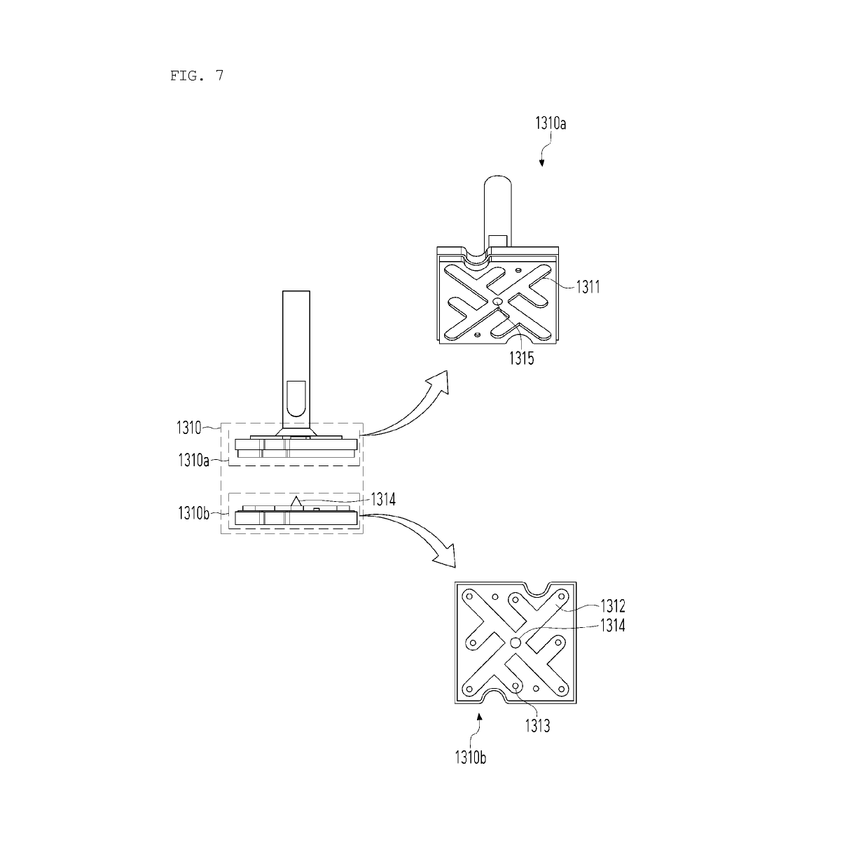

FIGS. 6A, 6B and 7 are exemplary views illustrating in detail the configuration of the cooling module 1300 illustrated in FIG. 3. Referring to FIGS. 6 and 7, the cooling module 1300 may include a cooling plate 1310, an inlet 1320, and/or a multiple outlets 1313.

In an embodiment, a guide groove 1330 may be formed in the cooling module 1300. In this case, the guide bars 1211 of the receptacle 1210 may be engaged with the guide grooves 1330, so the position of the cooling module 1300 may be aligned when the cooling module 1300 is accommodated in the receptacle 1210.

The inlet 1320 is a configuration for allowing the refrigerant injected by the handpiece 2000 to be introduced thereinto. The inlet 1320 may have a narrow and long cylindrical shape, and the refrigerant introduced into the inlet 1320 may pass through the cooling plate 1310 and then be discharged through the multiple outlets 1313.

The multiple outlets 1313 are a configuration for allowing the refrigerant introduced into the inlet 1320 to be sprayed therethrough into the first space S1. The multiple outlets 1313 may be configured as multiple holes arranged along a bottom surface of the cooling plate 1310 to spray the refrigerant to distributed positions in the first space S1. As illustrated in FIG. 6A and FIG. 6B, the multiple outlets 1313 may be arranged along the periphery of the bottom surface of the cooling plate 1310, but the scope of the present disclosure is not limited thereto. For example, the multiple outlets 1313 may be arranged along one or more concentric circles with different diameters on the bottom surface of the cooling plate 1310.

The cooling plate 1310 is a configuration for distributing the refrigerant introduced through the inlet 1320 to the multiple outlets 1313, and defines one or more flow paths between the inlet 1320 and the multiple outlets 1313. At this time, the flow paths may include multiple branches extending from a first point 1315 adjacent to the inlet 1320 respectively to the multiple outlets 1313 so that the refrigerant introduced through the inlet 1320 distributedly flows to the multiple outlets 1313. Here, the first point 1315 is a hole formed in the cooling plate 1310, and may be a hole connected to the inlet 1320.

The cooling plate 1310 and a flow path it defines will be described in more detail with reference to FIG. 7.

Referring to FIG. 7, a flow path structure of the cooling plate 1310 is illustrated. In an embodiment of FIG. 7, the cooling plate 1310 may include a first plate 1310a (i.e., an upper plate) having an inlet 1320 formed therein and a second plate 1310b (i.e., a lower plate) having multiple outlets 1313 formed therein.

In the present embodiment, the cooling plate 1310 may have one or more engraved branches 1311 on one of the first plate 1310a and the second plate 1310b and one or more corresponding embossed branches 1312 on the remaining plate to define a flow path through which fluid flow is possible. In FIG. 7, it is exemplified that the engraved branches 1311 are formed on the first plate 1310a and the embossed branches 1312 are formed on the second plate 1310b, but conversely, it is also possible to form the embossed branches 1312 on the first plate 1310a and the engraved branches 1311 on the second plate 1310b.

At this time, the width of the flow path may be adjusted by processing the depth of the engraved branches 1311 or the height of the embossed branches 1312 to vary. For example, when the depth of the engraved branches 1311 is made deeper, the width of the flow path may become widened, and when the depth of the engraved branches 1311 is made shallower, the width of the flow path may become narrowed. Similarly, when the height of the embossed branches 1312 is made higher, the width of the flow path may become narrowed, and when the height of the embossed branches 1312 is made lower, the width of the flow path may become widened.

The multiple outlets 1313 may be arranged at respective ends of the multiple branches 1312 formed on the second plate 1310b. Accordingly, the refrigerant introduced through the first point 1315 via the inlet 1320 may flow along the flow path (i.e., the multiple branches) inside the cooling plate 1310, and then be distributed to the multiple outlets 1313 located at the ends of the branches and discharged toward the first space.

In an embodiment, the first plate 1310a or the second plate 1310b may include a distribution member 1314 that distributes the refrigerant introduced through the inlet 1320 into the multiple branches. For example, the distribution member 1314 may be provided at a position corresponding to the first point 1315 of the first plate 1310a, or at a position on the second plate 1310b facing the first point 1315.

However, for the sake of simplicity of explanation, it is assumed in FIG. 7 that the distribution member 1314 is provided on the second plate 1310b.

Referring to FIG. 7, the distribution member 1314 is formed at a position on the second plate 1310b facing the first point 1315. At this time, the distribution member 1314 may be configured such that a first end thereof faces the first point 1315 and the first end has a narrower cross-sectional area than a second end of the distribution member 1314. In this case, a side profile of the distribution member 1314 may have an inclined surface. The refrigerant introduced through the first point 1315 may flow along the inclined surface of the distribution member 1314, enabling the refrigerant to be more easily distributed to each of the branches 1311 and 1312 of the flow path.

In an embodiment, the distribution member 1314 may have a conical or polygonal pyramid shape.

According to the configuration of the cooling module 1300, since the refrigerant is uniformly sprayed onto the first space S1 through the multiple outlets 1313, heat generated in the electrode module 1400 may be more effectively reduced, and uniform cooling may be provided for the entire cooling target area.

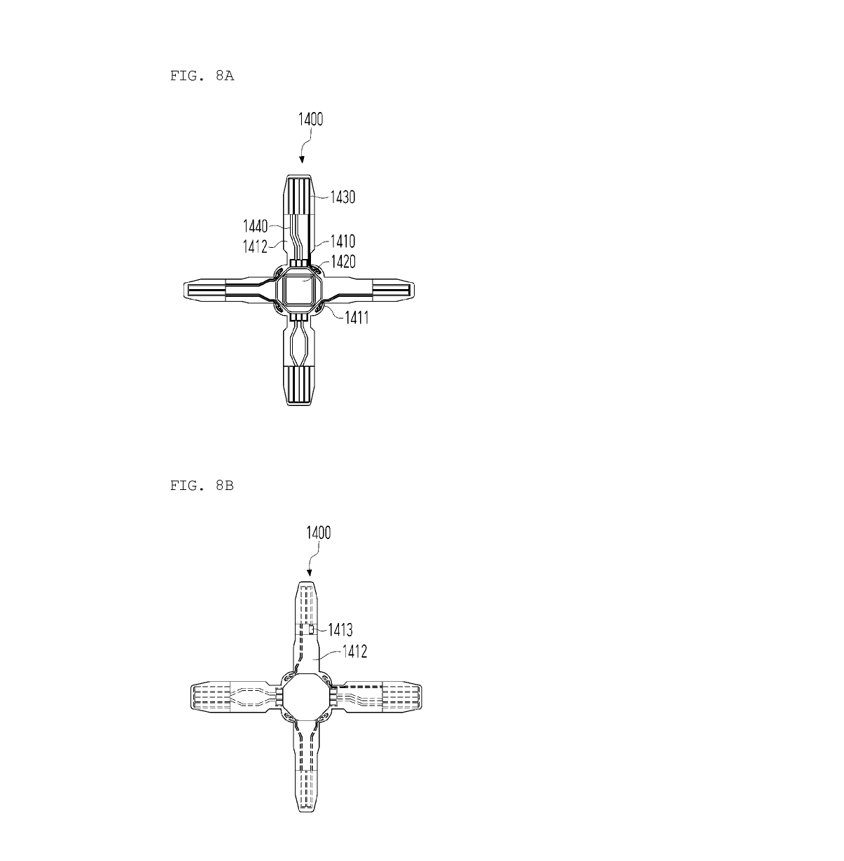

FIG. 8A and FIG. 8B are an exemplary view illustrating in detail the configuration of the electrode module 1400 illustrated in FIG. 3. FIG. 8A illustrates a first surface on which an electrode 1420 of the electrode module is located, and FIG. 8B illustrates a second surface, which is the back surface of the first surface.

Referring to FIG. 8A and FIG. 8B, the electrode module 1400 may include a substrate 1410 and the electrode 1420 formed on the substrate 1410.

The substrate 1410 may include an electrode region 1411 located centrally and one or more legs 1412 extending from the electrode region 1411.

The electrode 1420 may be formed on the electrode region 1411. The electrode 1420 may output the electrical energy transmitted from the handpiece 2000 toward the human skin in the form of electromagnetic waves or electric current.

In an embodiment, the electrode 1420 may be exposed to the outside of the housing 1100.

The one or more legs 1412 may be made of a flexible material, and may be fitted to the frame 1200, 1500 to secure the electrode module 1400 to the frame 1200, 1500.

Each of the one or more legs 1412 may be provided at an end thereof with a terminal 1430 that is electrically connected to the handpiece 2000 and receives the electrical energy provided by the handpiece 2000. One or more wires 1440 may be formed between the terminal 1430 and the electrode 1420 so as to transmit the electrical energy received by the terminal 1430 to the electrode 1420.

In one embodiment, the electrode module 1400 may include one or more semiconductor chips 1413. The semiconductor chips 1413 may be controllers or storage devices. For example, the semiconductor chips 1413 may be controller chips that control one or more sensors provided on the electrode region 1411, or memory chips that store information of the tip 1000 (e.g., ID or number of shots, etc.).

The one or more semiconductor chips 1413 may be provided at various positions on the substrate 1410. For example, as illustrated in FIG. 8A and FIG. 8B, the semiconductor chips 1413 may be located on the second surface, which is the back surface of the first surface where the electrode 1420 is located.

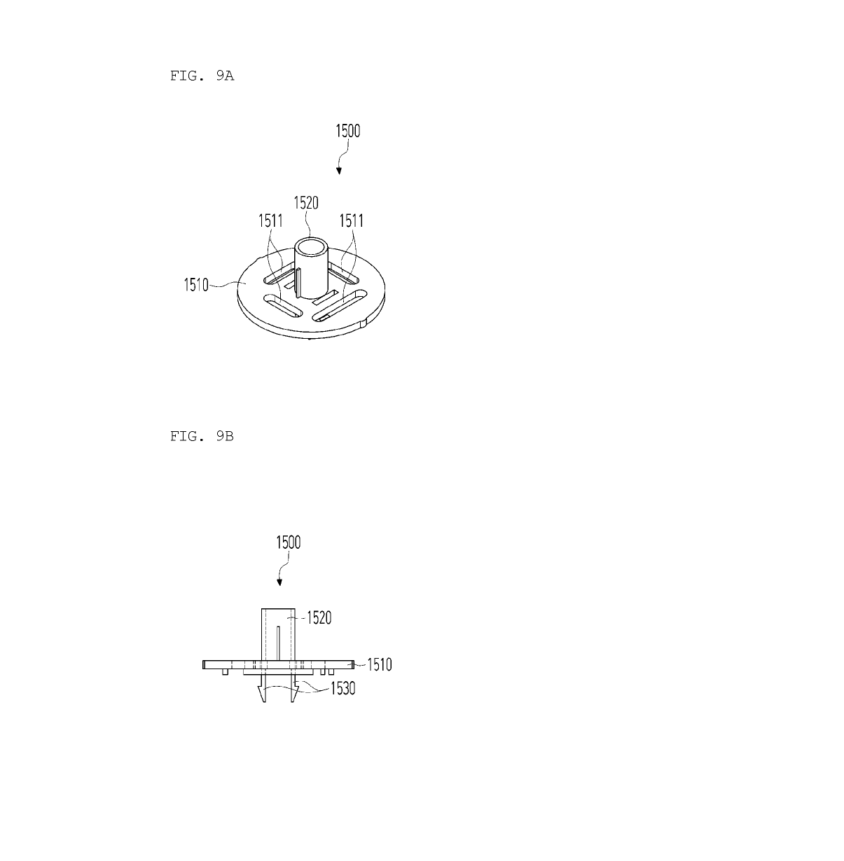

FIG. 9A and FIG. 9B are an exemplary view illustrating in detail the configuration of the cover member 1500 illustrated in FIG. 3.

Referring to FIG. 9A and FIG. 9B, the cover member 1500 may include a cover plate 1510, one or more connection holes 1511, and an inlet guide 1520.

The cover plate 1510 forms a body of the cover member 1500.

The one or more connection holes 1511 are perforated holes for exposing the terminals 1430 of the electrode module 1400 fitted to the frame 1200, 1500, and may be formed on the cover plate 1510.

The inlet guide 1520 is a configuration for guiding the position of the inlet 1320 of the cooling module 1300. The inlet 1320 of the cooling module 1300 may be connected to the refrigerant injection port of the handpiece 2000 through the inlet guide 1520 or inside the inlet guide 1520.

In an embodiment, a cover fastening portion 1530 for fastening the cover member 1500 to the base member 1200 may be provided on a bottom surface of the cover plate 1510. For example, a cover fastening portion 1530 in the form of a hook or protrusion may be provided, and the cover member 1500 and the base member 1200 may be fastened to each other in such a manner that the cover fastening portion 1530 is engaged with the frame fastening portion 1221 of the base member 1200.

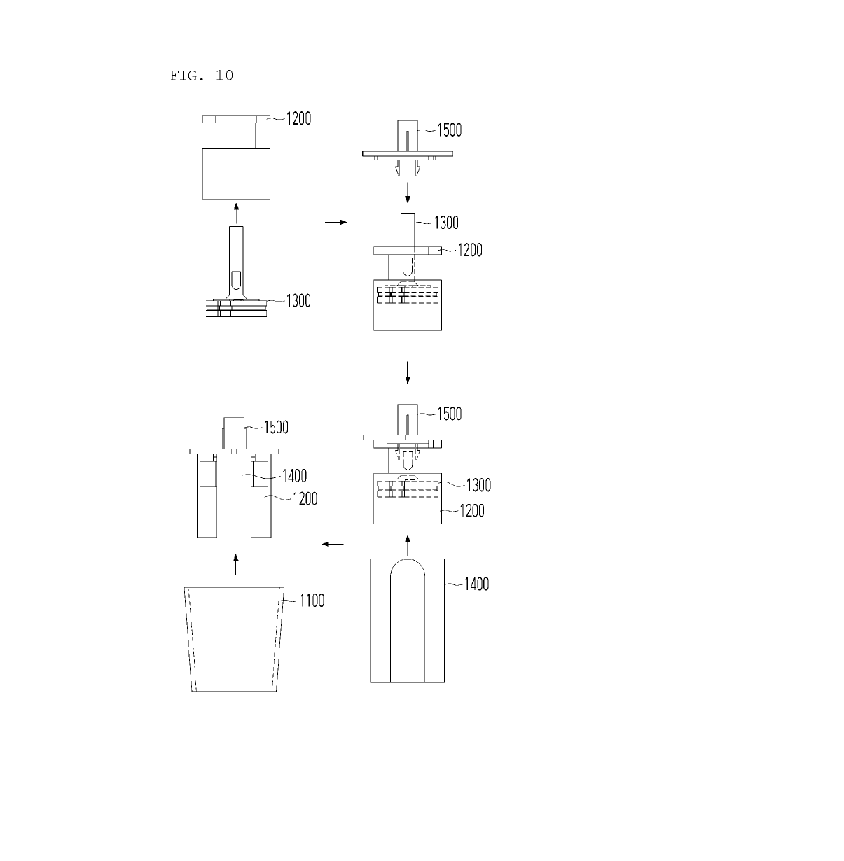

FIG. 10 is a view illustrating an exemplary assembly method of the tip 1000 according to an embodiment of the present disclosure.

Referring to FIG. 10, first, the cooling module 1300 is inserted into the receptacle 1210 of the base member 1200. Then, in that state, the cover member 1500 is coupled to the cooling module 1300 at a position facing the cooling module 1300. At this time, the inlet 1320 of the cooling module 1300 is inserted into the inlet guide 1520 of the cover member 1500, and the positions of the cover member 1500, the base member 1200, and the cooling module 1300 are aligned with each other.

Then, the electrode module 1400 is fitted and coupled to the frame 1200, 1500. After fitting engagement, the first space S1 is defined by the receptacle 1210 of the base member 1200 and the electrode region 1411 of the electrode module 1400. For example, a space surrounded by the receptacle 1210 and the electrode region 1411 serves as the first space S1.

Finally, the housing 1100 is coupled to the frame 1200, 1500 to complete the assembly of the tip 1000.

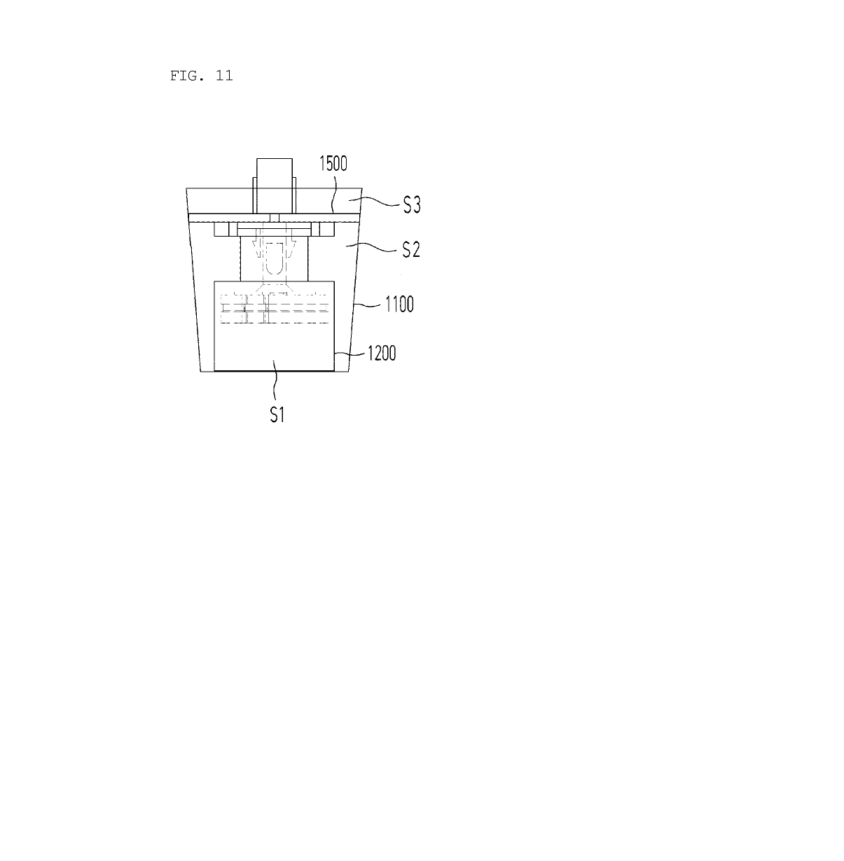

FIG. 11 is a view illustrating an internal space of the tip 1000, in which the internal space is partitioned by the main components 1100, 1200, and 1500 of the tip 1000. In FIG. 11, for the sake of convenience of explanation, the one or more legs 1412 of the electrode module 1400 are not illustrated.

The internal space of the tip 1000 may include a first space S1, a second space S2, and/or a third space S3.

The first space S1 is a space that is at least partially partitioned by the base member 1200, and may be, for example, a space surrounded by the receptacle 1210 and the electrode region 1411. The first space S1 may be a space where the refrigerant is sprayed by the cooling module 1300.

The second space S2 is a space that is partitioned by the frame 1200, 1500 and the housing 1100, and may be, for example, a space between the frame 1200, 1500 and the housing 1100. The second space S2 may be communicated with the first space S1 under predetermined conditions. For example, when the receptacle 1210 has a perforated hole, the refrigerant located in the first space S1 may flow to the second space S2 through the perforated hole.

The third space S3 is a space that is partitioned by the cover member 1500 and the housing 1100, and may be, for example, a space oriented toward the outside of the cover member 1500. The third space S3 is an open space when the tip 1000 is removed from the handpiece 2000, but when the tip 1000 is coupled to the handpiece 2000, the outside of the third space S3 is closed by a coupling surface of the handpiece 2000, thus becoming a closed space. The third space S3 may be communicated with the second space S2 under predetermined conditions. For example, when the cover plate 1510 has a perforated hole or undercut, the refrigerant located in the second space S2 may flow to the third space S3 through the perforated hole or undercut.

Hereinafter, a description will be given of various embodiments of forming a fluid channel for fluid communication between the spaces S1, S2, and S3 within the tip 1000, or for fluid communication between the spaces S1, S2, and S3 within the tip 1000 and an external space.

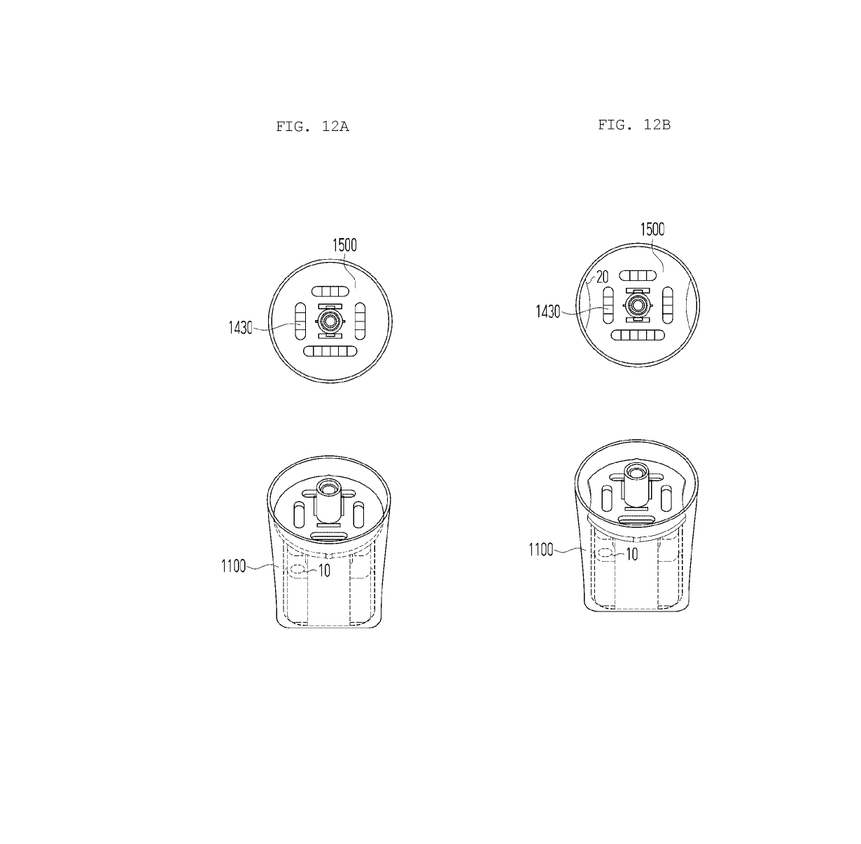

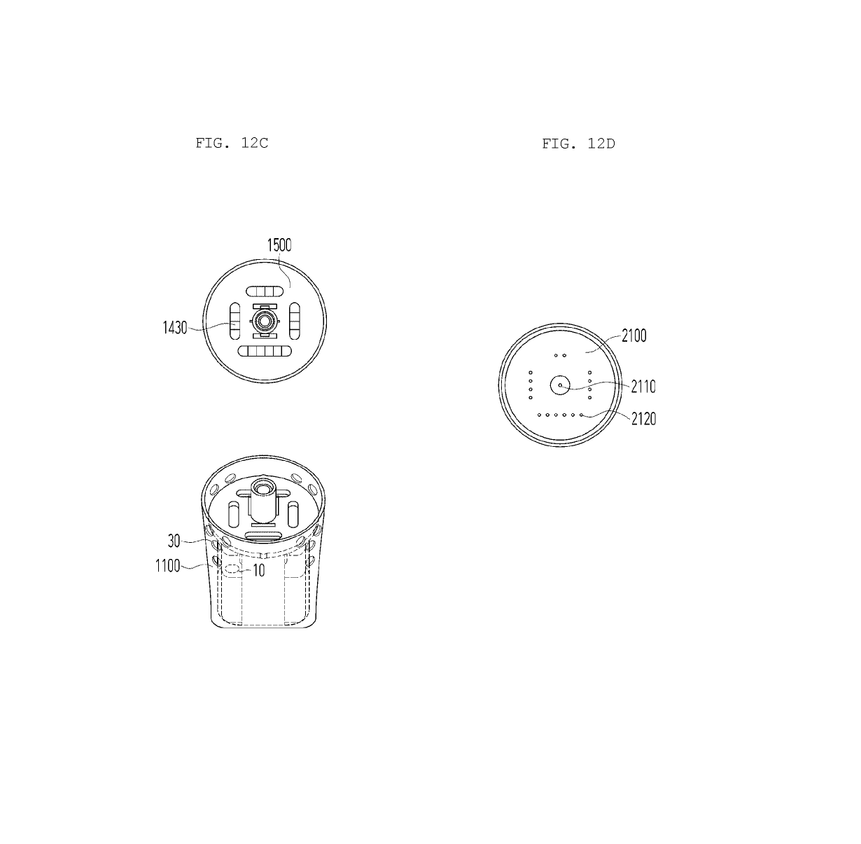

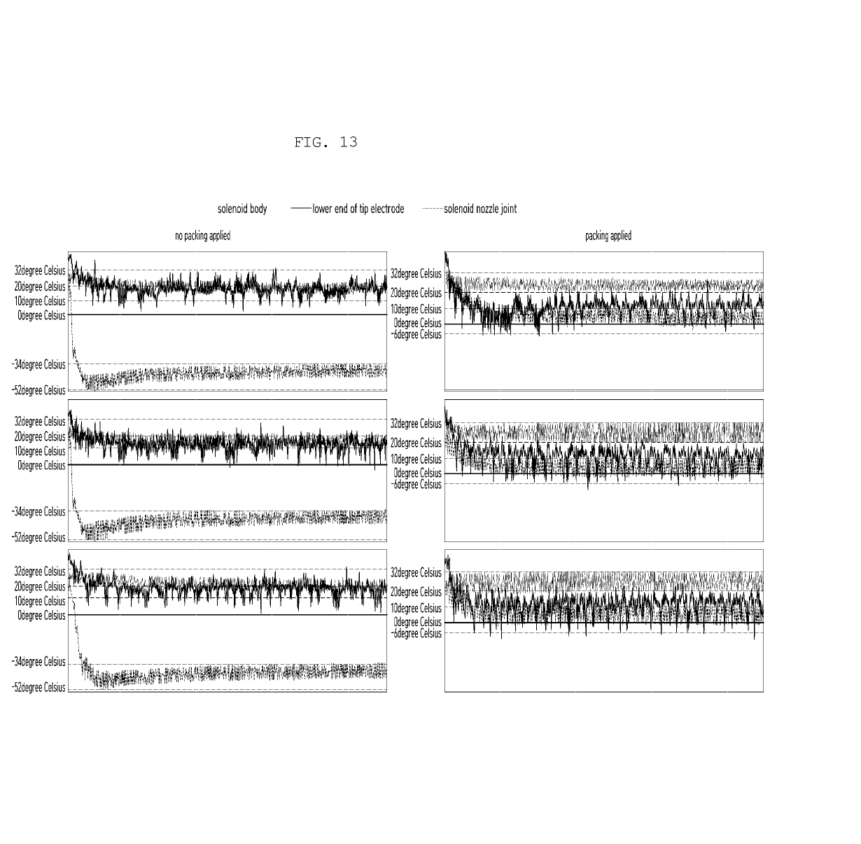

FIGS. 12A, 12B, 12C and 12D are a view illustrating various embodiments in which a fluid channel is formed inside the tip 1000 to more effectively remove a residual refrigerant inside the tip 1000.

FIG. 12A illustrates an embodiment in which a fluid channel is formed only in the base member 1200. For example, a perforated hole may be formed as a first channel 10 in the receptacle 1210 of the base member 1200. In this case, the first space S1 and the second space S2 may be communicated with each other through the first channel 10.

This is to ensure that the refrigerant sprayed into the first space S1 evaporates or is discharged quickly after spraying, thereby preventing any residual refrigerant from remaining in the first space S1. For example, when the first space S1 is a closed space, the refrigerant sprayed into the first space S1 may still remain in the first space S1 even after cooling, and may liquefy over time and accumulate in a liquid state inside the tip 1000. In this case, the refrigerant accumulated inside the tip 1000 may leak to the outside through an assembly surface or a gap of the tip 1000, and fall on the skin or face of the patient, causing discomfort or unexpected accidents during treatment.

Accordingly, by forming the first channel 10 in the base member 1200 to allow the first space S1 to be communicated with the second space S2, the sprayed refrigerant may flow into the second space S2 and thus may more easily evaporate or be discharged to the outside.

FIG. 12B illustrates an embodiment in which a second channel 20 is formed in the cover member 1500 together with the first channel 10 of the base member 1200. The second channel 20 may be formed, for example, by undercutting a portion of the cover plate 1510. However, this is exemplary and the scope of the present disclosure is not limited thereto. For example, the second channel 20 may be formed, for example, by forming a perforated hole in a portion of the cover plate 1510.

In FIG. 12B, the second space S2 is communicated with the third space S3 through the second channel 20. Accordingly, the refrigerant sprayed into the first space S1 may flow into the second space S2 through the first channel 10 and then be communicated again with the third space S3, enabling the refrigerant to flow in a wider space. This further promotes evaporation of the residual refrigerant.

FIG. 12C illustrates an embodiment in which a third channel 30 is formed in the housing 1100 together with the first channel 10 of the base member 1200. The third channel 30 may be formed, for example, by forming one or more perforated holes in the body portion 1110 of the housing 1100. In this case, the second space S2 or the third space S3 may be communicated with the outside of the housing 1100 through the third channel 30. Accordingly, the refrigerant sprayed into the first space S1 may flow into the second space S2 through the first channel 10 and then be communicated again with the outside of the housing 1100 through the third channel 30, enabling the refrigerant to more easily evaporate or be directly discharged to the outside of the housing 1100.

The embodiment of FIG. 12C may have additional beneficial effects in addition to removal of the residual refrigerant. For example, the residual refrigerant discharged to the outside of the housing 1100 through the third channel 30 may flow along an outer wall of the housing 1100 to reach the treatment site of the patient, thereby having the effect of directly cooling the treatment site. Therefore, it is possible to simultaneously cool the electrode 1420 inside the tip 1000 and directly cool the treatment site (e.g., the patient’s skin) outside the tip 1000.

Meanwhile, FIG. 12D illustrates a coupling surface 2100 of the handpiece 2000 coupled to the tip 1000. Referring to FIG. 12D, the coupling surface 2100 of the handpiece 2000 may be provided with a refrigerant injection port 2110 that is connected to the inlet 1320 of the cooling module 1300 to supply the refrigerant to the cooling module 1300 and a conductive connector 2120 that is in contact with the terminal 1430 of the electrode module 1400 to supply the electrical energy to the terminal 1430.

In an embodiment, the conductive connector 2120 may be a spring pin connector, such as a pogo pin.

Meanwhile, a packing member (not illustrated) may be applied between the refrigerant injection port 2110 and the inlet 1320. The packing member is a member that is fitted into the refrigerant injection port 2110 and/or the inlet 1320 to maintain airtightness between the refrigerant injection port 2110 and the inlet 1320, and may be made of synthetic resin, metal, fabric, and/or wood.

Maintaining airtightness between the refrigerant injection port 2110 and the inlet 1320 through the packing member may be very essential. For example, in the case where the packing member is not provided, when injecting the refrigerant, the refrigerant may leak from a connection point between the refrigerant injection port 2110 and the inlet 1320, resulting in loss of the refrigerant and a reduction in cooling effect. Additionally, the leaked refrigerant may flow to the coupling surface 2100 of the handpiece 2000 and cause damage to a product, such as freezing surrounding components.

FIG. 13 is a graph illustrating the results of an experiment comparing the difference between the case where a packing member is applied and the case where no packing member is applied.

Referring to the graph of FIG. 13, in the case where no packing member is applied, the temperature at a lower end of a tip electrode is 10 to 32 degree Celsius, so sufficient cooling is not achieved, while the temperature of a solenoid nozzle joint, i.e., the coupling surface 2100 of the handpiece 2000, is -35 to -52 degree Celsius, thereby freezing the components of the coupling surface 2100.

On the contrary, in the case where a packing member is applied, the refrigerant is completely delivered to the tip, so the temperature at the lower end of the tip electrode is -6 to 10 degree Celsius, thereby significantly improving the cooling effect, and the temperature of the solenoid nozzle joint, i.e., the coupling surface 2100 of the handpiece 2000, is maintained at 0 to 10 degree Celsius, thereby preventing the components of the coupling surface 2100 from freezing.

According to the embodiments of the present disclosure described so far, it is possible to provide an effective cooling structure capable of cooling a tip or a treatment site during treatment with a high frequency output device.

Additionally, by spraying the refrigerant in a distributed manner to a high-temperature area of the tip, it is possible to increase a cooling effect and uniformly lower the temperature of the tip.

Additionally, by providing one or more channels that communicate spaces inside the tip or communicate the inside of the tip with the outside of the tip, it is possible to quickly evaporate or discharge a residual refrigerant inside the tip, thereby effectively preventing a phenomenon in which the residual refrigerant liquefies and accumulates inside the tip.

Additionally, by applying a packing member between a handpiece and the tip, it is possible to further improve the cooling effect of the tip, and prevent freezing from occurring at a joint between the tip and the handpiece.

Although preferred embodiments of the present disclosure have been described with reference to the accompanying drawings, it will be apparent to those skilled in the art that the present disclosure may be embodied in specific forms other than those set forth herein without departing from the spirit and essential characteristics of the present disclosure. Therefore, the above embodiments should be construed in all aspects as illustrative and not restrictive. The scope of the disclosure should be determined by the appended claims and their legal equivalents, and all changes coming within the meaning and equivalency range of the appended claims are intended to be embraced therein.

What is claimed is:

1. A tip couplable to a handpiece, the tip comprising:
a housing:
a frame coupled to the housing;
an electrode module coupled to the frame and outputting electrical energy of a predetermined frequency; and
a cooling module spraying a refrigerant into a first space at least partially partitioned by the frame,
wherein the electrode module comprises:
a substrate; and
an electrode formed on a region of the substrate and outputting the electrical energy toward an outside of the housing,
wherein the refrigerant is introduced from the handpiece into the cooling module and then sprayed toward the region of the substrate.

2. The tip of claim 1, wherein the cooling module comprises:
an inlet allowing the refrigerant to be introduced thereinto;
multiple outlets allowing the refrigerant introduced into the inlet to be sprayed into the first space; and
a cooling plate defining a flow path between the inlet and the multiple outlets,
wherein the flow path comprises multiple branches extending from a first point adjacent to the inlet respectively to the multiple outlets so that the refrigerant introduced through the inlet distributedly flows to the multiple outlets.

3. The tip of claim 2, wherein the cooling plate comprises:
a first plate having the inlet formed therein;
a second plate having the multiple outlets formed therein; and
a distribution member distributing the refrigerant into the multiple branches.

4. The tip of claim 3, wherein the distribution member is formed on the first plate or the second plate, and
the distribution member is configured such that a first end thereof faces the first point and has a narrower cross-sectional area than a second end of the distribution member.

5. The tip of claim 4, wherein the distribution member has a conical or polygonal pyramid shape.

6. The tip of claim 1, wherein the frame comprises:
a base member at least partially surrounding the first space; and
a cover member coupled to the base member,
wherein a second space is formed between the frame and the housing, and
a first channel is formed in the base member to communicate the first space and the second space with each other.

7. The tip of claim 6, wherein a third space is formed between the frame and the handpiece, and
a second channel is formed in the cover member to communicate the second space and the third space with each other.

8. The tip of claim 6, wherein a third channel is formed in the housing,
wherein the third channel communicates the second space or the third space with the outside of the housing.

9. The tip of claim 1, wherein the substrate comprises:
one or more legs extending from the region,
wherein each of the one or more legs comprises:
a terminal electrically connected to the handpiece and receiving electrical energy from the handpiece; and
a wire electrically connecting the terminal to the electrode.

10. The tip of claim 9, wherein the one or more legs are made of a flexible material, and are fitted to the frame to secure the electrode module to the frame.

11. A high frequency output device, comprising:
a main body;
a handpiece connected to the main body; and
a tip coupled to the handpiece and receiving electrical energy from the handpiece and outputting electrical energy toward human body tissue while being coupled to the handpiece,
wherein the tip comprises:
a housing:
a frame coupled to the housing;
an electrode module coupled to the frame and outputting electrical energy of a predetermined frequency; and
a cooling module spraying a refrigerant into a first space at least partially partitioned by the frame,
wherein the electrode module comprises:
a substrate; and
an electrode formed on a region of the substrate and outputting electrical energy toward the human body tissue,
wherein the refrigerant is introduced from the handpiece into the cooling module and then sprayed toward the region of the substrate.

12. A handpiece for a high frequency output device, the handpiece comprising:
a tip;
an electrical circuit providing electrical energy of a predetermined frequency to the tip; and
a controller controlling an operation of the electric circuit,
wherein the tip comprises:
a housing:
a frame coupled to the housing;
an electrode module coupled to the frame and outputting electrical energy of a predetermined frequency; and
a cooling module spraying a refrigerant into a first space at least partially partitioned by the frame,
wherein the electrode module comprises:
a substrate; and
an electrode formed on a region of the substrate and outputting electrical energy toward an outside of the housing,
wherein the refrigerant is introduced from the handpiece into the cooling module and then sprayed toward the region of the substrate.
